(19) United States
(12) Patent Application Publication
(10) Pub. No.: US 2023/0355175 A1
(43) Pub. Date: Nov. 9, 2023

(54) HEADSET APPARATUS

(71) Applicant: University of Houston, Houston, TX (US)

(72) Inventors: Jose Contreras-Vidal, Houston, TX (US); Jeff Feng, Houston, TX (US)

(21) Appl. No.: 18/143,935

(22) Filed: May 5, 2023

Related U.S. Application Data
(60) Provisional application No. 63/364,214, filed on May 5, 2022.

(51) Int. Cl. A61B 5/00; A61B 5/256

(52) U.S. Cl. CPC A61B 5/6803; A61B 5/256; A61B 5/6831; A61B 5/265

(57) ABSTRACT

A headset device is described herein comprising a lower band comprising an outer surface and an inner surface, wherein a front portion of the lower band comprises at least one sensor positioned on the inner surface, wherein at least one arm extends from the lower band, wherein a proximal end of the at least one arm is rotatably attached to the lower band, wherein a distal end of the at least one arm comprises a sensor, wherein a rear portion of the lower band comprises adjustable straps for adjusting a circumference of the lower band. The headset device comprises an upper band comprising an upper surface and a lower surface, wherein at least one dry electrode component extends from the lower surface of the upper band, wherein the upper band is adjustably attached to the lower band.

### RELATED APPLICATIONS

This application claims the benefit of U.S. Application No. 63/364,214, filed May 5, 2022.

### TECHNICAL FIELD

The disclosure herein involves a headset device for positioning of electroencephalogram (EEG) and electrooculography (EOG) sensors on the head of a human subject.

### BACKGROUND

With the technology and signal processing advancements in recent years, mobile brain-body imaging (MoBI) systems are transformed into much more ambulatory devices, which opens up a potential for the advancement of the ecological validity of brain imaging research and more practical solutions for in-home medical monitoring and brain-computer interface (BCI) applications, as well in consumer electronics applications. With an increasing interest and demand in applying EEG scans in real-world environments, MoBI systems are developed to record brain dynamics during different tasks in the medical and non-medical fields. Even though there is an uptrend of developing commercial headsets in BCI-related research, consumer-like user-friendly headsets are still rare. Most of the commercially available portable systems are relatively expensive, require proprietary software to function, and lack flexibility or modularity. Ergonomically, headsets are not designed to be truly easy and intuitive to use. They often require trained technicians to help to put on the headset and operate the system. There is a growing need for a low-cost MoBI headset that can be set up with user-friendly ergonomics that is easy to operate and performs a quality scan and data collection consistently. Studies show most headsets on the market often do not fit as well as soft EEG caps. Headsets with a poor fit to the user will likely lose scanning signals due to the unstable sensor-skin contact and shifting position while in use. To date, traditional EEG caps are still the best in terms of accommodating both size and shape variation. There is a need for an easy to use one-hand operated headset that provides a custom fit for all users.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A headset device is described herein comprising under an embodiment a lower band comprising an outer surface and an inner surface, wherein a front portion of the lower band comprises at least one sensor positioned on the inner surface, wherein at least one arm extends from the lower band, wherein a proximal end of the at least one arm is rotatably attached to the lower band, wherein a distal end of the at least one arm comprises a sensor, wherein a rear portion of the lower band comprises adjustable straps for adjusting a circumference of the lower band. The headset device comprises an upper band comprising an upper surface and a lower surface, wherein at least one dry electrode component extends from the lower surface of the upper band, wherein the upper band is adjustably attached to the lower band.

A method is described herein under an embodiment comprising configuring a headset device for detection of electrical signals, wherein the headset device includes a lower band and upper band, wherein the lower band comprising an outer surface and an inner surface, wherein a front portion of the lower band comprises at least one sensor positioned on the inner surface, wherein a rear portion of the lower band comprises adjustable straps for adjusting a circumference of the lower band, wherein the upper band comprises an upper surface and a lower surface, wherein the upper band is adjustably attached to the lower band. The method includes configuring a first coupling of at least one dry electrode component to the upper band, wherein the first coupling comprises the at least one dry electrode component extending from the lower surface of the upper band. The method includes configuring a second coupling of at least one sensor arm to the lower band, wherein the second coupling comprises the at least one sensor arm extending from the lower band, wherein a proximal end of the at least one sensor arm is rotatably attached to the lower band, wherein a distal end of the at least one sensor arm comprises a sensor.

### BRIEF DESCRIPTION OF THE DRAWINGS

FIG. 1 shows a perspective view of a headset device, under an embodiment.

FIG. 2 shows a rear view of a headset device, under an embodiment.

FIG. 3 shows a front view of a headset device under an embodiment.

FIG. 4 shows a right side view of a headset device, under an embodiment.

FIG. 5 shows a left side view of a headset device, under an embodiment.

FIG. 6 shows a top view of a headset device, under an embodiment.

FIG. 7 shows a bottom view of a headset device, under an embodiment.

FIG. 8 shows a cross sectional view of a dry electrode component, under an embodiment.

FIG. 9 shows a cross sectional view of a dry electrode component, under an embodiment.

FIG. 10A shows a top view of a dry electrode component, under an embodiment.

FIG. 10B shows side view of a dry electrode component, under an embodiment.

FIG. 10C shows a side view of a dry electrode component, under an embodiment.

FIG. 10D show a perspective view of a dry electrode component, under an embodiment.

FIG. 11A shows a top view of a dry electrode assembly, under an embodiment.

FIG. 11B shows a side view of a dry electrode assembly, under an embodiment.

FIG. 11C shows a side view of a dry electrode assembly, under an embodiment.

FIG. 11D shows a perspective view of a dry electrode assembly, under an embodiment.

FIG. 12A shows a top view of a holder, under an embodiment.

FIG. 12B shows a side view of a holder, under an embodiment.

FIG. 12C shows a side view of a holder, under an embodiment.

FIG. 12D shows a perspective view of a holder, under an embodiment.

FIG. 13A shows a top view of a dry electrode assembly residing within a holder, under an embodiment.

FIG. 13B shows a side view of a dry electrode assembly residing within a holder, under an embodiment.

FIG. 13C shows a side view of a dry electrode assembly residing within a holder, under an embodiment.

FIG. 13D shows a perspective view of a dry electrode assembly residing within holder, under an embodiment.

FIG. 14 shows an exploded view of the headset device, under an embodiment.

FIGS. 15A-15D show a dry electrode component, under an embodiment.

FIGS. 16A-16D show a holder of a dry electrode component, under an embodiment.

FIGS. 17A-17D show a dry electrode assembly, under an embodiment.

FIGS. 18A-18D show a dry electrode assembly positioned within a holder, under an embodiment.

FIGS. 19A-19C show a cap attached to a holder, under an embodiment.

FIGS. 20A-20D show a holder of a dry electrode component, under an embodiment.

FIGS. 21A-21D show a proximal cylindrical body of a dry electrode assembly, under an embodiment.

FIGS. 22A-22D show a cap of a dry electrode component, under an embodiment.

### DETAILED DESCRIPTION

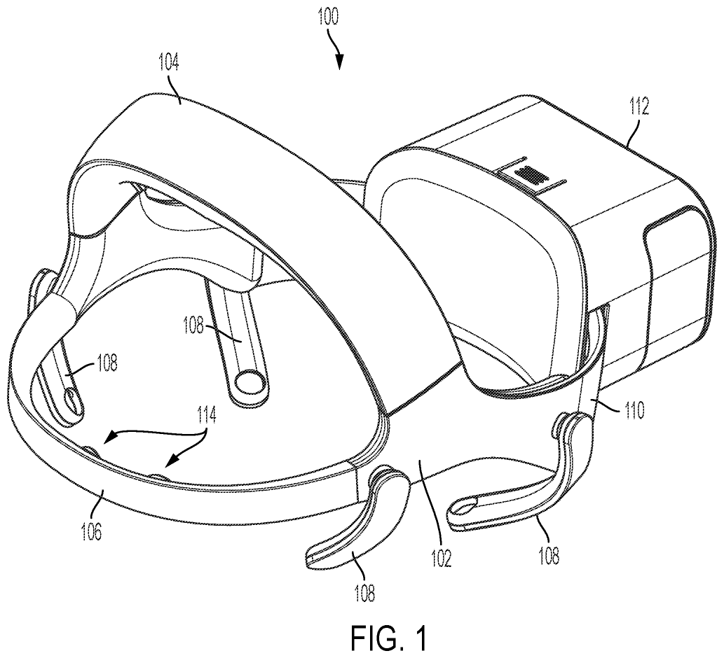

FIG. 1 shows a perspective view of a headset device 100. The headset comprises a lower band 102 and an upper band 104. The lower band encircles the head of a subject while the upper band extends over the top of the head. The upper band may be designed to pass across anterior (frontal), central (as shown) or posterior areas of the skull. A front portion 106 of the lower band passes around the forehead while a rear portion 110 of the lower band terminates in a casing 112 positioned at the rear of the head.

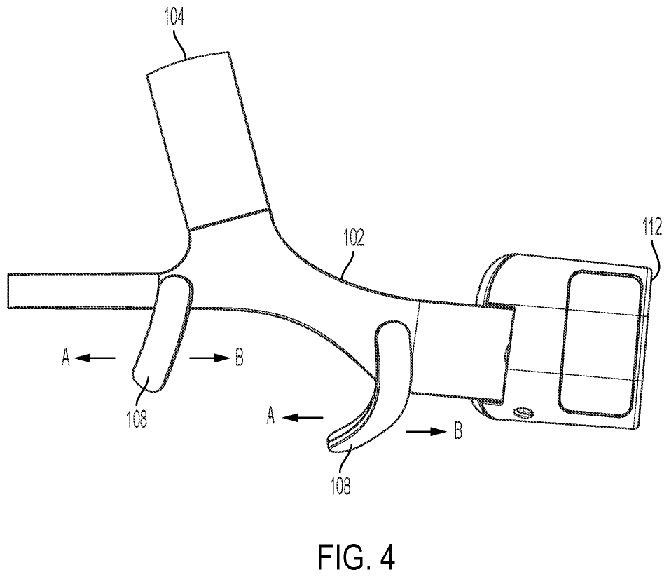

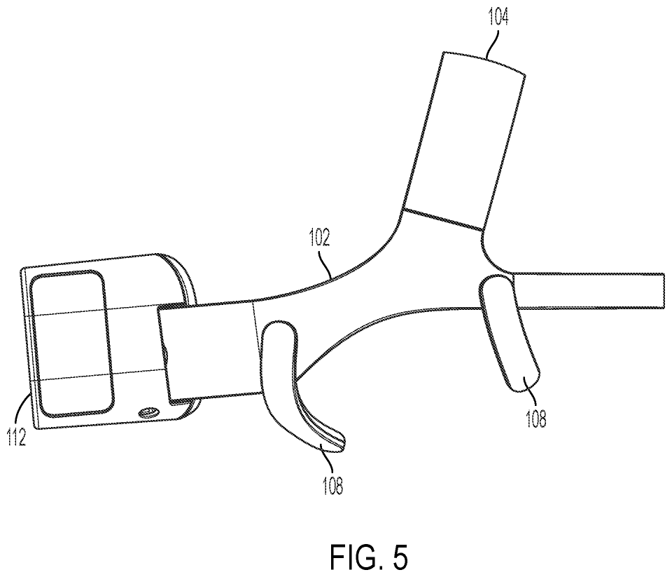

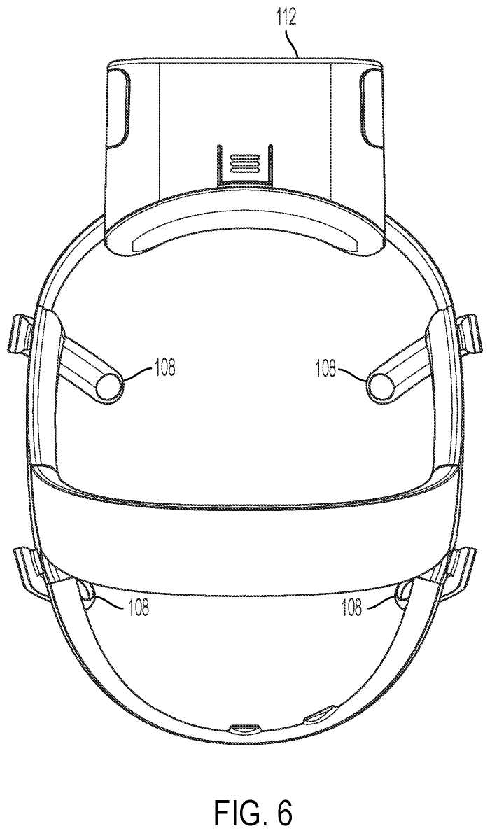

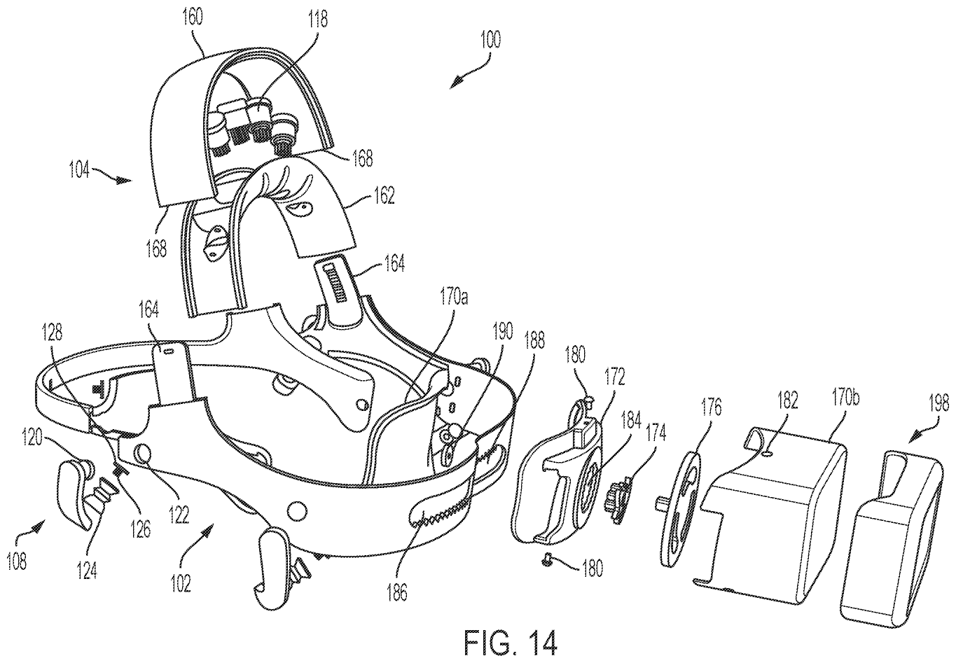

The inner side of the front portion 106 of the lower band positions one electrooculogram (EOG) sensor 114 along the forehead to measure electrical signals generated by eye blinks and eye movements. (Note that element number 114 in FIG. 1 shows potential locations of such sensor. Further additional EOG sensors may be placed along the interior of the lower band's front portion). The sensor is under one embodiment a flat snap EEG/ECG/EOG electrode with Silver/Silver Chloride (Ag/AgCl) Coating manufactured by Florida Research Institute, Cocoa Beach, FL. The lower band features four arms 108 that extend in a downward direction. The proximal end of each arm is attached to the lower band while the distal end features either an EOG or reference sensor. (Under the embodiment shown in FIGS. 1 and 14, the arms attached to the front portion of the lower band feature EOG sensors while the arms attached to the rear portion of the lower band feature reference/ground sensors). As seen in FIG. 14 a protrusion 120 at the proximal end of each arm 108 is secured by press fit through a receiving hole 122 in the lower band. The distal end of each arm 108 is attached to a sensor positioner 124 which receives a securing post 126 of a sensor 128. Once secured to the lower band 102, the arm is rotatable around the axis of attachment. As seen in FIG. 4, the arms may rotate laterally in directions A and B. The rotatable coupling of each arm allows a wide range of flexibility in placement of distally located sensors.

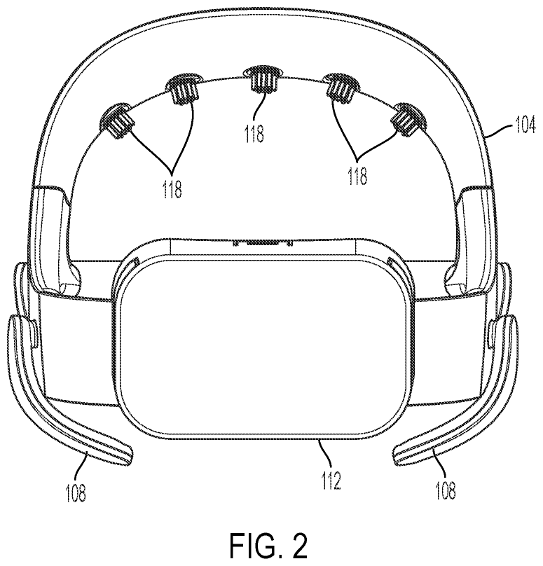

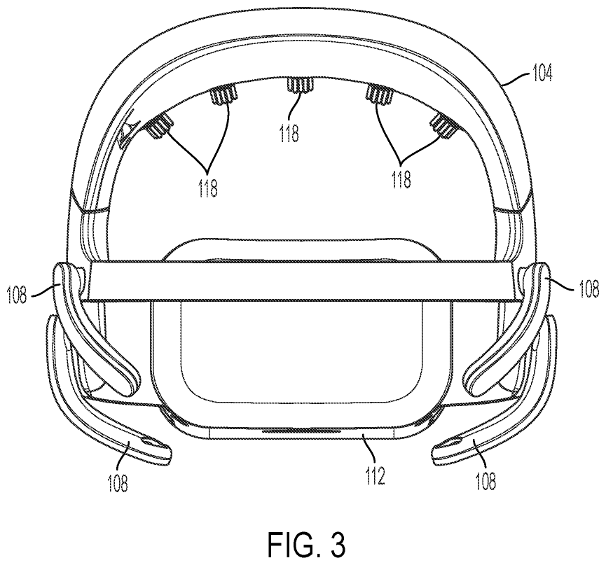

As seen in FIGS. 2, 3, 7, and 14, the upper band 104 features five dry EEG electrode components (as described in greater detail below). The electrodes are under one embodiment a Spike snap EEG electrode with a Silver/Silver Chloride Coating manufactured by Florida Research Institute. Alternative embodiments may provide for additional or fewer dry electrode components. FIG. 14 shows an exploded view of the device 100. The view of FIG. 14 illustrates that the dry electrode components 118 are positioned between an upper portion 160 and lower portion 162 of the upper band 104. When the upper portion and lower portion are secured together (in a snap fit), the dry electrode components extend towards the subject's head. Note that the curvature of the upper band fixes the downwardly extending electrodes along a path that matches the curvature of the subject's head.

The lower band 102 is adjustably attached to the upper band 104. As seen in FIG. 14, the lower band attaches to the upper band using a tongue 164 and groove 168 configuration. A tongue component 164 extending from the lower band is received by groove 168 in component 160 of the upper band. FIG. 1 shows the upper band in a minimal peripheral distance configuration, i.e., tongue component 164 is completely received within the groove 168. The tongue/groove attachment allows adjustable separation of the upper band from the lower band thereby increasing or decreasing the peripheral distance of the upper band.

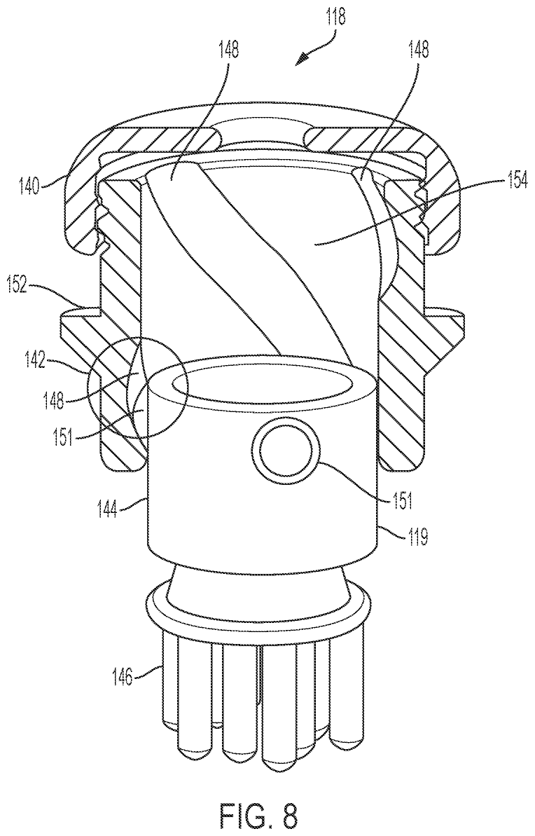

FIG. 8 shows a cross sectional view of a dry electrode component 118. The dry electrode component comprises a cap 140, a holder 152, and a dry electrode assembly 119. The dry electrode assembly 119 includes a distal spike element 146 and proximal cylindrical body 144. (Note that the spike element 146 may have other form factors without changing the functional aspects of the dry electrode assembly 119). The cap is threadably attached to the holder. The holder comprises an annular structure which receives the dry electrode assembly 119. The proximal cylindrical body 144 holds the spike element 146. The inner surface of the holder features three helical threads 148. The proximal cylindrical body 144 features three protrusion buttons 151. Each button tracks a thread as best demonstrated 142 in FIG. 8. As the dry electrode assembly 119 moves in a proximal and distal direction, the button/thread configuration rotates the dry electrode assembly and therefore the distally located spike element.

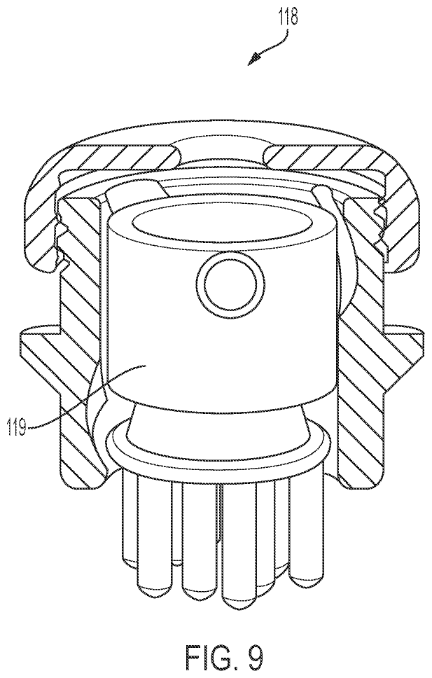

FIG. 8 shows the dry electrode assembly 119 in a fully extended position. FIG. 9 shows the dry electrode assembly 119 in a fully retracted position. Under an embodiment, a compression spring is located in the space 154 between an upper surface of the dry electrode assembly 119 and the cap 140 which biases the dry electrode assembly towards a fully extended position. Therefore, the dry electrode assembly 119 remains in extended position when not in use. In operation, a user places the helmet device on user's head. The user's head then urges the dry electrode assembly 119 in a proximal direction as the helmet device is seated. This proximally directed force causes buttons (or protrusions) 151 to slide along corresponding helical grooves 148 resulting in angular rotation of the dry electrode assembly 119 including the spike element 146. The angular rotation of the dry electrode assembly tunnels a pathway through a user's hair to ensure contact between electrical contacts and scalp in the device's seated position. The spring biases the dry electrode assembly 119 towards an extended position to ensure continued contact during use. In operation, the dry electrode assembly 119 translates perpendicularly from the skull surface with a translation range of 10 mm. In other words, the range of the dry electrode assembly's proximal and distal motion is 10 mm. Alternative embodiments implement shorter or longer distances depending on application or to accommodate varying hair styles.

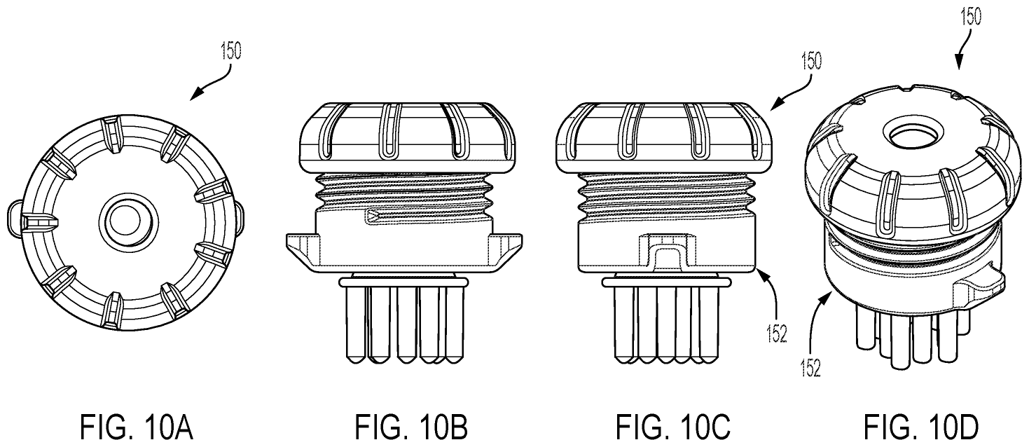

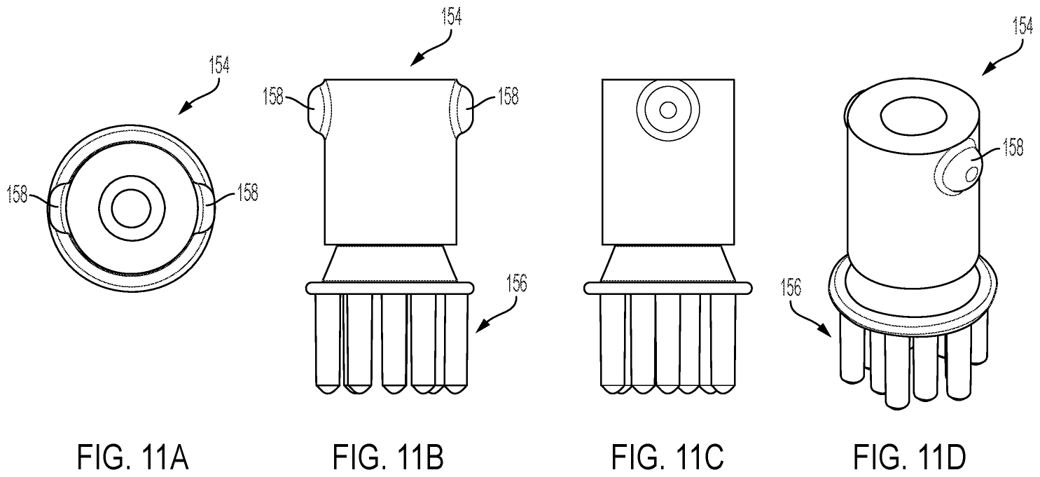

FIGS. 10A-13D show an electrode component under an alternative embodiment. FIGS. 10A-10D show a cap 150 threadably attached to a holder 152. FIGS. 11A-11D illustrate dry electrode assembly 154 which comprises a proximal cylindrical body and distal spike element 156. As opposed to the electrode shown in FIGS. 8 and 9, the proximal cylindrical component features two protrusion buttons 158.

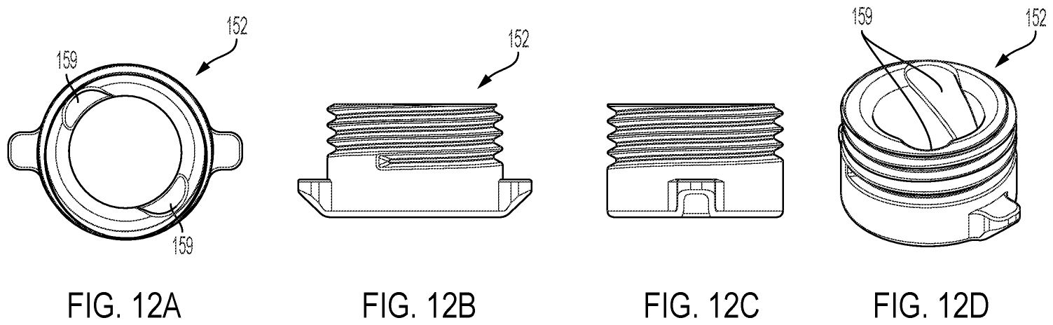

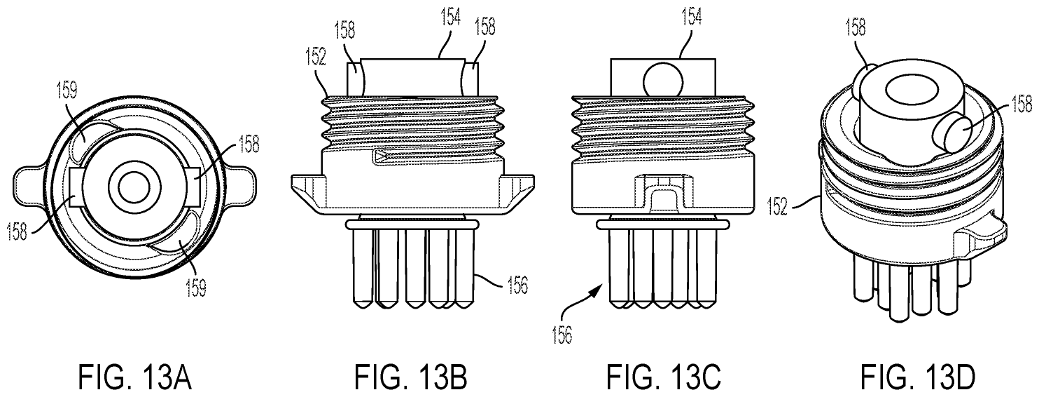

FIGS. 12A-12D show the holder 152. The inner surface of the holder features two helical threads 159. Each corresponding button tracks a thread as the dry electrode moves in a proximal and distal direction thereby rotating the dry electrode assembly 154 and the distally located electrode spike element. FIGS. 13A-13D show the dry electrode assembly 154 positioned within the holder 152. Note that the protrusion buttons are offset from the helical threads for purposes of illustration.

As indicated above the rear portion 110 of the lower band 102 terminates within a housing 112. The housing includes separable front and rear components 170a and 170b. FIG. 14 shows the two components 170a and 170b separated thereby revealing the interior components of the housing. The interior components include a gear bracket 172, a gear 174, and adjustment wheel 176. The rear component 170b attaches directly to the gear bracket 172. Through holes 182 located on an upper and lower surface of the rear component 170b receive screws 180 that threadably attach to corresponding screw bosses on an upper and lower surface of the gear bracket 172. When the rear compartment is in a secured position, the wheel 176, gear 174, and bracket 172 collapse upon each other. A post extending from the wheel engages a receiving hole in the gear in a press or interference fit. The post terminates at a stopper component 190 which resides between the front component 170a and gear bracket 172. Rotation of the wheel 176 then rotates gear 174.

As shown in FIG. 14, the rear portion of 110 of lower band 102 comprises two adjustable straps which feature openings 186, 188. A receiving hole 184 in gear bracket 172 receives and locates gear 174 in a position for engaging teeth cut into openings 186, 188 of the adjustable straps. Note that opening 186 features teeth cut along its lower edge. A corresponding opening 188 features teeth cut along its upper edge. Front component 170a snap fits onto gear bracket 172 and secures the adjustable straps and corresponding openings 186, 188 against an interior surface of the gear bracket 172. The attached front component 170a secures the openings in an overlapping configuration. When the front compartment 170a and the rear compartment 170b are attached to the gear bracket 172, the receiving hole 184 of the gear bracket and the openings 186, 188 receive gear 174 such that teeth of gear 174 simultaneously engage lower teeth of opening 186 and upper teeth of opening 188.

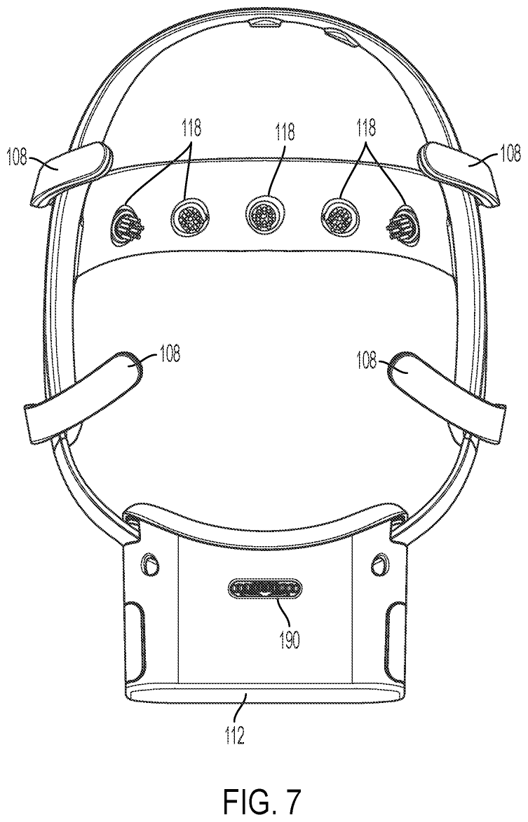

A lower portion of wheel 176 extends through an opening (see FIG. 7, 190) on a lower surface of the housing 112. In operation a user rotates the wheel to adjust the circumference of the lower band 102. As the user adjusts the wheel clockwise, rotation of gear 174 extends the straps outwardly in opposing directions thereby increasing the circumference of lower band 102. As the user adjusts the wheel counterclockwise, rotation of gear 174 extends the straps inwardly in opposing directions thereby decreasing the circumference of lower band 102.

The housing is fit with a rear component 198 under an embodiment. The rear component serves both decorative and protective purposes. Under an embodiment, the rear component includes circuitry coupled to the sensors (as deployed by the headset device and as described above). The circuitry is configured to receive, store, and/or transmit sensor data.

The headset device may transmit information to remote systems, computing devices, or other components through on or more communication paths. The communication paths may include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths may include removable fixed mediums like floppy disks, hard disk drives, and CD-ROM disks, as well as flash RAM, Universal Serial Bus (USB) connections, RS-232 connections, telephone lines, buses, and electronic messaging.

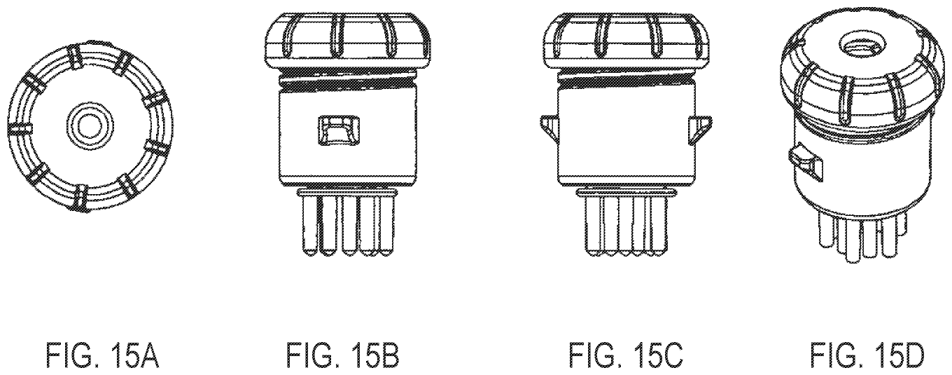

FIGS. 15A-15D show a dry electrode component, under an embodiment.

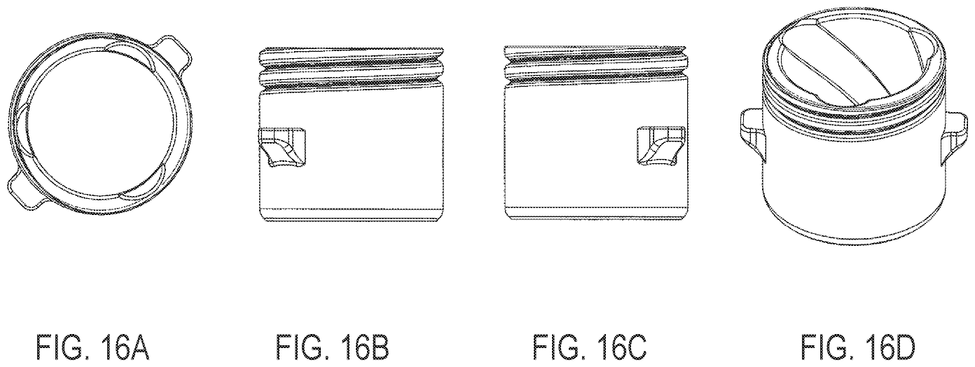

FIGS. 16A-16D show a holder of a dry electrode component, under an embodiment.

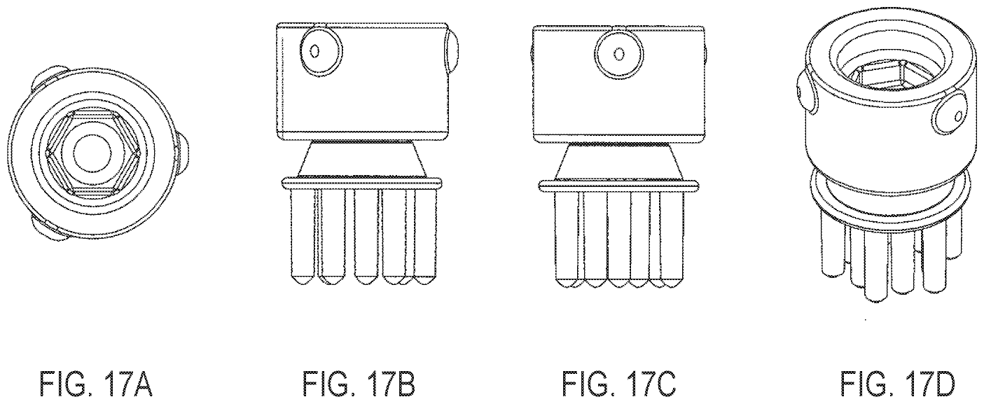

FIGS. 17A-17D show a dry electrode assembly, under an embodiment.

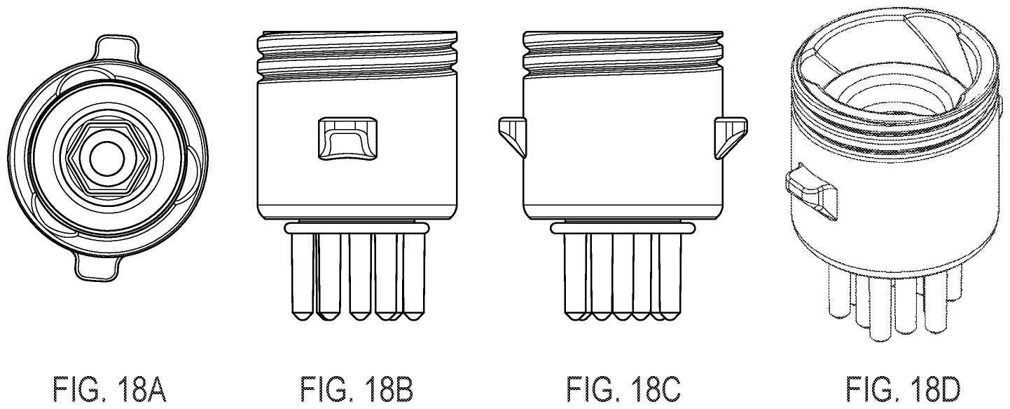

FIGS. 18A-18D show a dry electrode assembly positioned within a holder, under an embodiment.

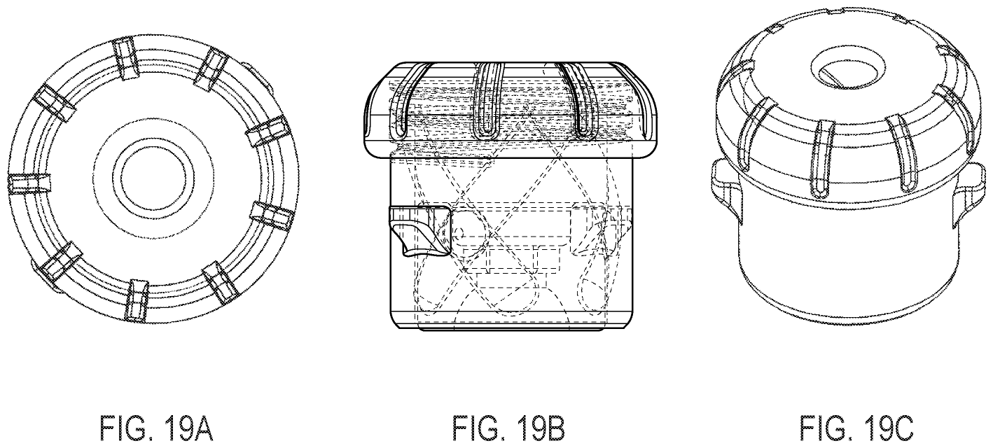

FIGS. 19A-19C show a cap attached to a holder, under an embodiment.

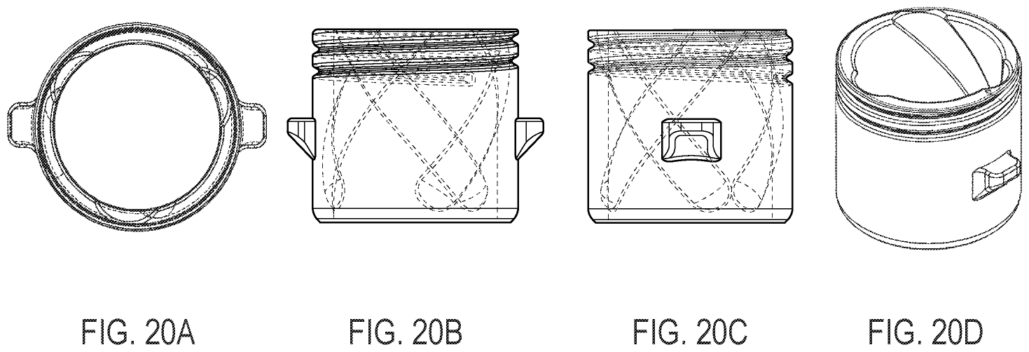

FIGS. 20A-20D show a holder of a dry electrode component, under an embodiment.

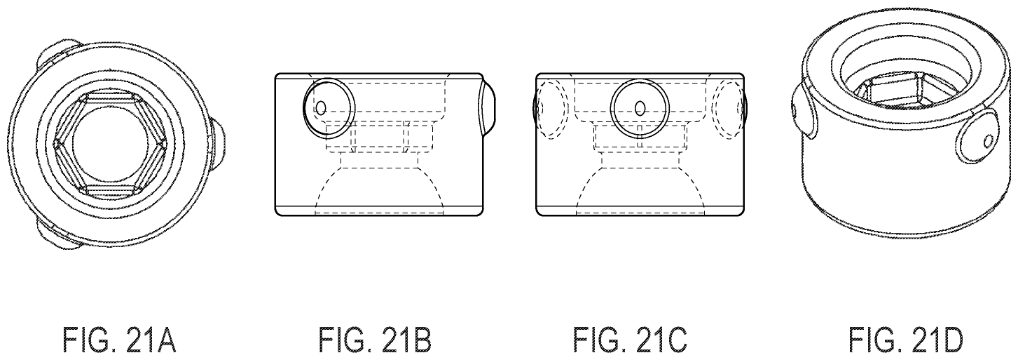

FIGS. 21A-21D show a proximal cylindrical body of a dry electrode assembly, under an embodiment.

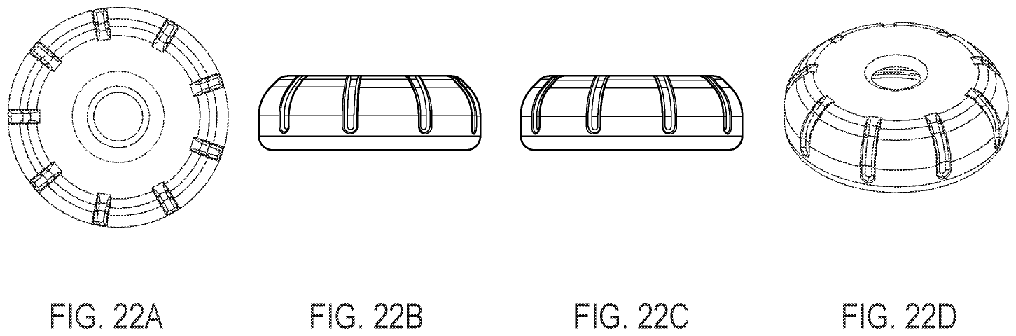

FIGS. 22A-22D show a cap of a dry electrode component, under an embodiment.

The above description of the headset device is not intended to be exhaustive or to limit the device the precise forms disclosed. While specific embodiments are described herein for illustrative purposes, various equivalent modifications are possible within the scope of the disclosed embodiment, as those skilled in the relevant art will recognize.

A headset device is described herein comprising under an embodiment a lower band comprising an outer surface and an inner surface, wherein a front portion of the lower band comprises at least one sensor positioned on the inner surface, wherein at least one arm extends from the lower band, wherein a proximal end of the at least one arm is rotatably attached to the lower band, wherein a distal end of the at least one arm comprises a sensor, wherein a rear portion of the lower band comprises adjustable straps for adjusting a circumference of the lower band. The headset device comprises an upper band comprising an upper surface and a lower surface, wherein at least one dry electrode component extends from the lower surface of the upper band, wherein the upper band is adjustably attached to the lower band.

The at least one dry electrode component comprises a holder and a dry electrode assembly, under an embodiment.

A proximal end of the dry electrode assembly comprises radially extending protrusions, under an embodiment.

An interior surface of the holder comprises helical grooves, under an embodiment.

Each protrusion tracks a corresponding helical groove, under an embodiment.

Reciprocating motion of the dry electrode assembly causes angular motion of the dry electrode assembly as the protrusions track the corresponding helical grooves, under an embodiment.

The adjustable straps comprise a first strap and a second strap, under an embodiment.

The first strap comprises a first opening and the second strap comprises a second opening, under an embodiment.

The first opening and the second opening overlap, under an embodiment.

Teeth of a gear simultaneously engages teeth cut into a lower edge of the first opening and teeth cut into an upper edge of the second opening, under an embodiment.

Clockwise rotation of the gear extends the first strap and the second strap outwardly in opposing directions thereby increasing the circumference of lower band, under an embodiment.

Counter-clockwise rotation of the gear retracts the first strap and the second strap inwardly in opposing directions thereby decreasing the circumference of lower band, under an embodiment.

The adjustable attachment comprises a tongue and groove configuration, under an embodiment.

The adjustable attachment provides a configurable peripheral length of the upper band, under an embodiment.

A method is described herein under an embodiment comprising configuring a headset device for detection of electrical signals, wherein the headset device includes a lower band and upper band, wherein the lower band comprising an outer surface and an inner surface, wherein a front portion of the lower band comprises at least one sensor positioned on the inner surface, wherein a rear portion of the lower band comprises adjustable straps for adjusting a circumference of the lower band, wherein the upper band comprises an upper surface and a lower surface, wherein the upper band is adjustably attached to the lower band. The method includes configuring a first coupling of at least one dry electrode component to the upper band, wherein the first coupling comprises the at least one dry electrode component extending from the lower surface of the upper band. The method includes configuring a second coupling of at least one sensor arm to the lower band, wherein the second coupling comprises the at least one sensor arm extending from the lower band, wherein a proximal end of the at least one sensor arm is rotatably attached to the lower band, wherein a distal end of the at least one sensor arm comprises a sensor.

The at least one dry electrode component comprises a holder and a dry electrode assembly, under an embodiment.

A proximal end of the dry electrode assembly comprises radially extending protrusions, under an embodiment.

An interior surface of the holder comprises helical grooves, under an embodiment.

Each protrusion tracks a corresponding helical groove, under an embodiment.

Reciprocating motion of the dry electrode assembly causes angular motion of the dry electrode assembly as the protrusions track the corresponding helical grooves, under an embodiment.

1. A headset device comprising,
a lower band comprising an outer surface and an inner surface, wherein a front portion of the lower band comprises at least one sensor positioned on the inner surface, wherein at least one arm extends from the lower band, wherein a proximal end of the at least one arm is rotatably attached to the lower band, wherein a distal end of the at least one arm comprises a sensor, wherein a rear portion of the lower band comprises adjustable straps for adjusting a circumference of the lower band;
an upper band comprising an upper surface and a lower surface, wherein at least one dry electrode component extends from the lower surface of the upper band, wherein the upper band is adjustably attached to the lower band.

2. The headset device of claim 1, wherein the at least one dry electrode component comprises a holder and a dry electrode assembly.

3. The headset device of claim 2, wherein a proximal end of the dry electrode assembly comprises radially extending protrusions.

4. The headset device of claim 3, wherein an interior surface of the holder comprises helical grooves.

5. The headset device of claim 4, wherein each protrusion tracks a corresponding helical groove.

6. The headset device of claim 5, wherein reciprocating motion of the dry electrode assembly causes angular motion of the dry electrode assembly as the protrusions track the corresponding helical grooves.

7. The headset device of claim 6, wherein a spring biases the dry electrode assembly towards an extended position.

8. The headset device of claim 1, wherein the adjustable straps comprise a first strap and a second strap.

9. The headset device of claim 8, wherein the first strap comprises a first opening and the second strap comprises a second opening.

10. The headset device of claim 9, wherein the first opening and the second opening overlap.

11. The headset device of claim 10, wherein teeth of a gear simultaneously engage teeth cut into a lower edge of the first opening and teeth cut into an upper edge of the second opening.

12. The headset device of claim 11, wherein clockwise rotation of the gear extends the first strap and the second strap outwardly in opposing directions thereby increasing the circumference of lower band.

13. The headset device of claim 12, wherein counterclockwise rotation of the gear retracts the first strap and the second strap inwardly in opposing directions thereby decreasing the circumference of lower band.

14. The headset device of claim 1, wherein the adjustable attachment comprises a tongue and groove configuration.

15. A method comprising,
configuring a headset device for detection of electrical signals, wherein the headset device includes a lower band and upper band, wherein the lower band comprising an outer surface and an inner surface, wherein a front portion of the lower band comprises at least one sensor positioned on the inner surface, wherein a rear portion of the lower band comprises adjustable straps for adjusting a circumference of the lower band, wherein the upper band comprises an upper surface and a lower surface, wherein the upper band is adjustably attached to the lower band;
configuring a first coupling of at least one dry electrode component to the upper band, wherein the first coupling comprises the at least one dry electrode component extending from the lower surface of the upper band;
configuring a second coupling of at least one sensor arm to the lower band, wherein the second coupling comprises the at least one sensor arm extending from the lower band, wherein a proximal end of the at least one sensor arm is rotatably attached to the lower band, wherein a distal end of the at least one sensor arm comprises a sensor.

16. The method of claim 15, wherein the at least one dry electrode component comprises a holder and a dry electrode assembly.

17. The method of claim 16, wherein a proximal end of the dry electrode assembly comprises radially extending protrusions.

18. The method of claim 17, wherein an interior surface of the holder comprises helical grooves.

19. The method of claim 18, wherein each protrusion tracks a corresponding helical groove.

20. The method of claim 19, wherein reciprocating motion of the dry electrode assembly causes angular motion of the dry electrode assembly as the protrusions track the corresponding helical grooves.
